(19) United States
(12) Patent Application Publication
(10) Pub. No.: US 2024/0164928 A1
(43) Pub. Date: May 23, 2024

(54) METHOD FOR SUTURING TWO PORTIONS OF A VESSEL TOGETHER

(71) Applicant: PALLIARE LIMITED, Galway (IE)

(72) Inventors: John O’DEA, Galway (IE); Hilary Elizabeth Barrett, Limerick (IE)

(73) Assignee: PALLIARE LIMITED, Galway (IE)

(21) Appl. No.: 18/355,552

(22) Filed: Jul. 20, 2023

(30) Foreign Application Priority Data
Jul. 22, 2022 (IE) S2022/0124

(51) Int. Cl. A61F 5/00

(52) U.S. Cl. CPC A61F 5/0086; A61F 5/0069; A61F 5/0076; A61F 5/0089

(57) ABSTRACT

An endoscopic sleeve gastroplasty procedure comprises forming a gastric sleeve (5) through a stomach (1) by suturing a first portion (9) of a wall (3) of the stomach (1) to a second portion (11) of the wall (3). First and second rows (20, 22) of first and second mounds (18, 19) are formed in the first and second portions (9,11) by injecting a viscus bulking solution (25) into a submucosal layer (28) at sites (17) of the wall (3) to form pockets (27) therein to form the first and second mounds (18, 19). Sutures (15) are inserted into respective pairs (16) of the first and second mounds (18, 19) with anchor bars (38) of the sutures (15) located in the corresponding pockets (27). The sutures (15) are tightened for drawing the first and second mounds (18, 19) together to in turn draw the first and second portions (9, 11) of the wall (3) of the stomach (1) together to form the gastric sleeve (5).

### FIELD OF THE INVENTION

The present invention relates to a method for suturing two portions of a wall of a vessel, lumen or organ together in the body of a human or animal subject. The invention also relates to a method for carrying out a sleeve gastroplasty procedure internally within the stomach of a human or animal subject.

The term “vessel” will hereinafter be used to include a vessel, a lumen or an organ, and the term “vessel wall” will be hereinafter used to refer to the wall of a vessel, a lumen or an organ.

### BACKGROUND TO THE INVENTION

In minimally invasive surgical procedures, and in particular in endoscopic procedures, where it is required to suture a vessel internally within the vessel, in general, a suturing device is inserted through an instrument channel of an endoscope into the vessel, and the suturing of the parts of the vessel is carried out internally within the vessel. For example, where it is desired to evert a polyp in a vessel, for example, in a colon of a human or animal subject, the colon adjacent opposite sides of the polyp sutured together in order to urge the polyp to appear on the outside of the colon where it may be excised from the external side of the colon. Additionally, in a sleeve gastroplasty procedure being carried out endoscopically in the stomach of a human or animal subject, a suturing instrument is inserted into the stomach through an instrument channel of the endoscope, and opposite sides of the stomach wall are sutured together in order to form a gastric sleeve extending through the stomach. In general, in such cases of everting a polyp in a colon or forming a gastric sleeve in a sleeve gastroplasty procedure in the stomach of a human or animal subject, sutures are used of the type which comprise an elongated suture thread terminating in respective opposite ends thereof in anchor elements, and a cinch clip is slideably located on a loop formed by the suture thread between the anchor elements for tightening the suture. The anchor elements of the suture are typically located in a distal pointed end of a cannula of a suturing instrument, and during the suturing of the parts of the colon or stomach together, the cannula must be urged sequentially through the wall of the colon or the stomach to deposit the anchor elements on the outer surface of the colon or stomach with the suture thread extending from the anchor elements through the colon or stomach wall into the colon or stomach, so that as the suture is being tightened by urging the cinch clip of the suture along the loop of the suture thread towards the anchor elements, the anchor elements are drawn together, to in turn draw the parts of the colon or vessel wall together.

A particular disadvantage of such suturing methods for suturing parts of a vessel together where the pointed tip of the cannula must be urged through a vessel wall from the inner surface of the vessel to an outer surface thereof to deposit the anchor elements of the suture adjacent the outer surface of the vessel wall, is that there is a high risk of a vessel, lumen or organ adjacent the vessel in which the procedure is being carried out being punctured by the tip of the cannula. This is a particularly serious risk, in the event of, for example, an artery being located adjacent the vessel in which the procedure is being carried out, since the puncturing of such an artery could have fatal consequences.

There is therefore a need for a method for suturing two parts of a vessel together, particularly in a minimally invasive surgical procedure, for example, a procedure carried out endoscopically which addresses this problem. There is also a need for a method for carrying out a sleeve gastroplasty procedure which addresses this problem, and in particular, there is a need for a method for carrying out an endoscopic sleeve gastroplasty procedure which addresses the problem.

The present invention is directed towards providing a method for suturing two parts of a vessel together, and in particular, though not limited to a method for suturing two parts of a vessel together carried out endoscopically. The invention is also directed towards a method for carrying out a sleeve gastroplasty procedure, and in particular, though not limited to a method for carrying out an endoscopic sleeve gastroplasty procedure.

### SUMMARY OF THE INVENTION

According to the invention there is provided a method for suturing a first portion of a wall of a vessel in a cavity in the body of a human or animal subject to a second portion of the wall of the vessel either interiorly in the interior of the vessel, or exteriorly of the vessel in the cavity, the method comprising:
forming at least one first mound on a site of the first portion of the vessel wall,
forming at least one second mound on a site of the second portion of the vessel wall,
each one of the first and second mounds being formed by forming a pocket in a submucosal layer of the vessel wall or between the submucosal layer and a muscular layer of the vessel wall adjacent the site at which the corresponding one of the first and second mounds is to be formed,
inserting at least one suture into or through a corresponding pair of the at least one first mound, and the at least one second mound, and
tightening the suture to draw the corresponding pair of the first and second mounds together.

In one embodiment of the invention each pocket is formed by injecting an injectable fluid into the submucosal layer of the vessel wall or between the submucosal layer and the muscular layer of the vessel wall where the pocket is to be formed.

In one embodiment of the invention the injectable fluid comprises an injectable liquid.

In another embodiment of the invention the injectable fluid comprises a viscous liquid, and preferably, an injectable viscous solution.

In another embodiment of the invention the viscous solution comprises a viscous bulking solution, and preferably, the viscous bulking solution comprises an injectable viscous bulking solution, and ideally, the viscous solution comprises a viscous gel.

In another embodiment of the invention the injectable fluid comprises a liquid of low viscosity.

In another embodiment of the invention the liquid of low viscosity comprises water or
a water
based solution, and preferably, a saline solution.

In another embodiment of the invention, the viscosity of the injectable fluid is sufficient to minimise dispersal of the injectable fluid from the corresponding pocket for a predefined time period at least sufficient to allow the suture to be inserted into the mound, and preferably, for a time period of at least 5 minutes, and advantageously, for a time period of at least 10 minutes, and preferably, for a time period of at least 15 minutes, and more preferably, for a time period of at least 20 minutes, and ideally for a time period of approximately 30 minutes.

In one embodiment of the invention the injectable fluid comprises a lifting agent.

In another embodiment of the invention the lifting agent comprises a lifting agent sold under the trade mark ORISE gel by Boston Scientific.

In another embodiment of the invention the injectable fluid comprises a lifting agent sold under the trade mark ELEVIEW by Aries Pharmaceutical.

In another embodiment of the invention the injectable fluid comprises a non-toxic fluid, and preferably, the injectable fluid comprises a biocompatible fluid.

In another embodiment of the invention the quantity of the injectable fluid injected into the submucosal layer or between the submucosal layer and the muscular layer of the corresponding one of the first and second portions of the vessel wall is sufficient to form the pocket of a size to form the corresponding one of the first and second mounds of the desired size.

In one embodiment of the invention the quantity of the injectable fluid injected into the submucosal layer or between the submucosal layer and the muscular layer of the corresponding one of the first and second portions of the vessel wall to form the pocket of the corresponding one of the first and second mounds lies in the range of 1 ml to 10 ml, and preferably, lies in the range of 1 ml to 5 ml, and more preferably, lies in the range of 1 ml to 2 ml.

In some embodiments of the invention when one or a pair of the mounds is formed as an elongated mound extending longitudinally along the one or both of the first and second portions of the wall of the vessel to be sutured together, the quantity of the injectable fluid injected into the submucosal layer or between the submucosal layer and the muscular layer to form each one of the corresponding pockets may be as much as 10 ml and greater.

In one embodiment of the invention a plurality of the first mounds are formed on the respective sites on the first portion of the vessel wall, and a plurality of the second mounds are formed on the respective sites on the second portion of the vessel wall.

Preferably, the first mounds are spaced apart on the first portion of the vessel wall, and advantageously, the second mounds are spaced apart on the second portion of the vessel wall.

In one embodiment of the invention the first mounds are equi-spaced apart on the first portion of the vessel wall, and preferably, the second mounds are equi-spaced apart on the second portion of the vessel wall.

Advantageously, the first mounds are formed in a first row on the first portion of the vessel wall, and preferably, the second mounds are formed in a second row on the second portion of the vessel wall. Preferably, the first and second mounds are spaced apart along the respective first and second rows.

In one embodiment of the invention the spacing between the first mounds is similar to the spacing of the second mounds.

In one embodiment of the invention the first and second mounds are formed sequentially on the first and second portions of the vessel wall.

In one embodiment of the invention each one of the first and second mounds comprises a substantially circular mound when viewed in plan, and preferably, each one of the first and second mounds is of diameter in the range of 3 mm to 25 mm, and advantageously, in the range of 5 mm to 20 mm, and more preferably, the diameter of each one of the first and second mounds lies in the range of 5 mm to 10 mm, and ideally, the diameter of each one of the first and second mounds is approximately 8 mm.

In an alternative embodiment of the invention each one of the first and second mounds comprises an elongated mound extending along the corresponding one of the first and second portions of the vessel wall, and preferably, each elongated first and second mound is formed by an elongated pocket formed in the submucosal layer or between the submucosal layer and the muscular layer of the vessel wall and extending along the corresponding one of the first portion and the second portion adjacent the site at which the elongated mound is to be formed.

In one embodiment of the invention the elongated pocket of the corresponding elongated mound is of tubular cross-section.

In another embodiment of the invention each elongated mound is of transverse width lying in the range of 3 mm to 25 mm, and preferably, lying in the range of 5 mm to 20 mm, and more preferably, lying in the range of 5 mm to 10 mm, and advantageously, each elongated mound is of transverse width of approximately 8 mm.

In another embodiment of the invention the height of each one of the first and second mounds from a surface of the vessel wall adjacent the site at which the mound is formed lies in the range of 5 mm to 10 mm, and preferably, the height of each one of the first and second mounds lies in the range of 6 mm to 8 mm, and preferably, the height of each one of the first and second mounds from the surface adjacent the site at which the mound is formed is approximately 7 mm.

In one embodiment of the invention one suture is inserted into or through each pair of the first and second mounds.

In an alternative embodiment of the invention plurality of the sutures are inserted into or through each pair of first and second mounds, and preferably, a plurality of the sutures are inserted into or through one of the first and second mounds when the one of the first or second mounds comprises an elongated mound.

In one embodiment of the invention each suture is urged into or through the first and second mounds of the corresponding pair thereof, and preferably, each suture is urged sequentially into or through the first and second mounds of the corresponding pair thereof.

In another embodiment of the invention each suture comprises a suture thread terminating at respective opposite ends thereof in respective anchor elements, the suture thread defining a loop formed by two legs of the suture thread extending from the respective anchor elements, the loop extending through a cinch clip, the cinch clip being urgeable along the loop towards the anchor elements for reducing the effective length of the suture thread extending between the anchor elements to tighten the suture.

In one embodiment of the invention the cinch clip of each structure is configured to be a tight sliding fit on the loop sufficient to resist movement of the cinch clip along the loop in a direction away from the anchor elements.

In one embodiment of the invention each anchor element comprises an anchor bar, and preferably, the suture thread extends from each anchor bar at a position intermediate the ends of the anchor bar, and preferably, the suture thread extends from each anchor bar at a location substantially midway between the ends of the bar.

In one embodiment of the invention each suture is inserted into the first and second mounds of the corresponding pair thereof by inserting the anchor elements into the pockets of the respective first and second mounds of the pair thereof with the legs of the suture thread extending from the anchor elements through the corresponding mounds.

In one embodiment of the invention the leg of the suture thread of each suture extending from the corresponding anchor element extends from the corresponding anchor element through the submucosal layer or a part thereof of the corresponding one of the first and second mounds of the corresponding pair thereof.

In one embodiment of the invention the anchor elements of each suture element are inserted sequentially into the pockets of the first and second mounds of the corresponding pair thereof.

In an alternative embodiment of the invention each suture is inserted into the first and second mounds of the corresponding pair thereof by urging the anchor elements of the suture through the respective ones of the first and second mounds from an entry side of the corresponding ones of the first and second mounds to an exit side thereof with the anchor elements located on the exit side of the corresponding ones of the first and second mounds and the legs of the suture thread extending from the anchor elements through the corresponding ones of the first and second mounds from the exit side thereof to and through the entry side thereof.

In another embodiment of the invention the anchor elements of each suture are inserted sequentially into or through the first and second mounds of the corresponding pair thereof.

In another embodiment of the invention on one or more of the sutures being inserted into or through the first and second mounds of the corresponding pair or pairs thereof, the or each suture is tightened by urging the cinch clip thereof along the loop thereof towards the anchor elements thereof for drawing the first and second mounds of the corresponding pair thereof together.

In another embodiment of the invention the sutures are inserted sequentially into or through the respective pairs of the first and second mounds.

Preferably, commencement of insertion of the sutures into or through the pairs of the first and second mounds is not commenced until all of the first and second mounds have been formed.

In another embodiment of the invention each suture is tightened by urging the cinch clip thereof along the loop formed by the suture thread thereof towards the anchor elements for drawing the first and second mounds of the corresponding pair thereof together.

In one embodiment of the invention each suture is tightened before the next suture to be inserted is inserted.

In another embodiment of the invention a plurality of the sutures are inserted into or through the corresponding pair or pairs of the first and second mounds before the sutures of the plurality thereof are tightened, and preferably, the sutures are tightened sequentially.

In a further embodiment of the invention the sutures are not tightened until all of the sutures have been inserted into or through the pair or pairs of the first and second mounds.

In another embodiment of the invention each suture is inserted into or through the first and second mounds of the pair thereof by inserting a cannula into or through each one of the first and second mounds of the pair thereof, and preferably, the suture is located in a bore of the cannula and the anchor elements of the suture are sequentially discharged from the cannula as the cannula is urged into or through the respective first and second mounds of the corresponding pair thereof.

In another embodiment of the invention the suturing of the first and second portions of the vessel is carried out endoscopically, and in one embodiment of the invention the suturing of the first and second portions of the vessel is carried out internally within the vessel, and advantageously, the endoscope is inserted into the vessel.

Alternatively, the suturing of the first and second portions of the vessel together is carried out externally of the vessel, and in one embodiment of the invention is carried out in the cavity in the body of the human or animal subject in which the vessel is located.

In another embodiment of the invention the method is carried out endoscopically by inserting the endoscope into the vessel or the cavity orally, rectally or vaginally.

In a further embodiment of the invention the first and second portions of the vessel wall of the vessel are first and second portions of the wall of a stomach, and preferably, the first and second portions of the wall of the stomach are sutured together in the carrying out of a sleeve gastroplasty procedure, and preferably, the endoscope is inserted orally into the subject to the stomach through the oesophagus.

The invention also provides a method for carrying out a sleeve gastroplasty procedure in the stomach of a human or animal subject in which first and second portions of the wall of the stomach are sutured together to form a gastric sleeve by the method according to the invention.

### Advantages of the Invention

The advantages of the invention are many. A particularly important advantage of the invention is that by injecting the injectable fluid solution at the respective sites for forming the first and second mounds, the first and second mounds are readily easily formed, and furthermore, all the first and second mounds may be formed prior to commencement of insertion of the sutures into the respective pairs of the first and second mounds. This has the advantage of minimising the number of times the various instruments must be withdrawn and re-entered into the stomach through an endoscope, and therefore, minimises the time required to carry out a sleeve gastroplasty procedure or any other procedure which requires suturing two parts of a wall of a vessel together.

By virtue of the fact that the first and second mounds are formed within the vessel on the inner surface of the wall thereof, the sutures are inserted into or through the first and second mounds, with the anchor elements located either in the pockets or on an exit side of the mounds. Thus, by tightening the sutures, the first and second mounds are drawn together, which in turn results in the first and second portions of the vessel similarly being drawn together in order to promote knitting of the first and second portions of the vessel together. Accordingly, there is no need for a cannula of a suturing instrument to be urged through the wall of the vessel adjacent the first and second portions thereof for depositing the anchor elements of the sutures on the respective outer sides of the wall of the vessel, which could result in the cannula as it exits the wall of the vessel puncturing an adjacent vessel, lumen or organ in the cavity within which the vessel is located, with potentially fatal consequences.

Another advantage of the invention is that before the commencement of suturing, the mounds may be formed along the respective portions of the vessel to be sutured together, thereby enabling a surgeon or a clinician to mark out the lines along which the portions of the vessel wall are to be sutured by the mounds before any suturing commences.

The invention will be more clearly understood from the following description of some non-limiting examples thereof which are given solely by way of example with reference to the accompanying drawings.

### BRIEF DESCRIPTION OF THE DRAWINGS

FIG. 1 is a cross-sectional perspective view of a portion of a stomach of a subject in which a sleeve gastroplasty procedure has been carried out endoscopically internally in the stomach of the subject by a method according to the invention,

FIG. 2 is a cross-sectional perspective view of the portion of the stomach of FIG. 1 during carrying out of the sleeve gastroplasty procedure by the method according to the invention,

FIG. 3 is a view similar to FIG. 2 of the portion of the stomach of FIG. 1 during a step in the sleeve gastroplasty procedure being carried out by the method according to the invention,

FIG. 4 is a diagrammatic view of the stomach of FIG. 1 during a step in the carrying out of the sleeve gastroplasty procedure by the method according to the invention,

FIG. 5 is a diagrammatic view of the stomach of FIG. 1 during a further step in the carrying out of the sleeve gastroplasty procedure by the method according to the invention,

FIG. 6 is a perspective view of a suture for use in the carrying out of the sleeve gastroplasty procedure in the stomach of FIG. 1 by the method according to the invention,

FIG. 7 is an enlarged cross-sectional view of a portion of the stomach of FIG. 1 during the carrying out of the sleeve gastroplasty procedure by the method according to the invention,

FIG. 8 is a cross-sectional view of another detail of the stomach of FIG. 1 during the carrying out of the sleeve gastroplasty procedure by the method according to the invention,

FIG. 9 is a view similar to that of FIG. 8 illustrating a further step during the carrying out of the sleeve gastroplasty procedure by the method according to the invention,

FIG. 10 is a view similar to that of FIG. 8 illustrating a further step during the carrying out of the sleeve gastroplasty procedure by the method according to the invention,

FIG. 11 is a cross-sectional top plan view of a portion of the stomach of FIG. 1 illustrating a further step during the carrying out of the sleeve gastroplasty procedure by the method according to the invention,

FIG. 12 is a view similar to that of FIG. 11 illustrating a further step during the carrying out of the sleeve gastroplasty procedure by the method according to the invention,

FIG. 13 is a view of a subject during the carrying out of the sleeve gastroplasty procedure in the stomach of FIG. 1 of the subject by the method according to the invention,

FIG. 14 is a cross-sectional perspective view of a portion of a stomach of a subject in which a sleeve gastroplasty procedure has been carried out endoscopically internally in the stomach of the subject by a method according to another embodiment of the invention,

FIG. 15 is a cross-sectional perspective view of the portion of the stomach of FIG. 14 during carrying out of the sleeve gastroplasty procedure by the method according to the embodiment of the invention of FIG. 14,

FIG. 16 is a perspective view of a detail of the stomach of FIG. 14 during the carrying out of the sleeve gastroplasty procedure by the method according to the embodiment of the invention of FIG. 14,

FIG. 17 is another perspective view of the detail of FIG. 16 of the stomach of FIG. 14 during the carrying out of the sleeve gastroplasty procedure by the method according to the embodiment of the invention of FIG. 14,

FIG. 18 is a cross-sectional view of another detail of a portion of the stomach of FIG. 14 during the carrying out of the sleeve gastroplasty procedure by the method according to the embodiment of the invention of FIG. 14, and,

FIG. 19 is a side elevational view of a detail similar to that of FIG. 18 of the stomach of FIG. 14 during the carrying out of the sleeve gastroplasty procedure by the method according to the embodiment of the invention of FIG. 14.

### DETAILED DESCRIPTION OF THE INVENTION

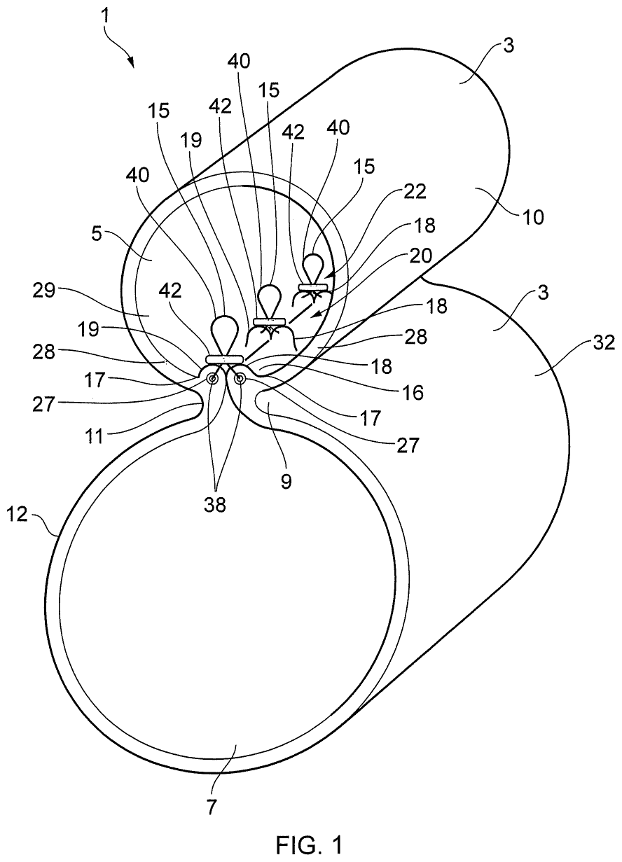

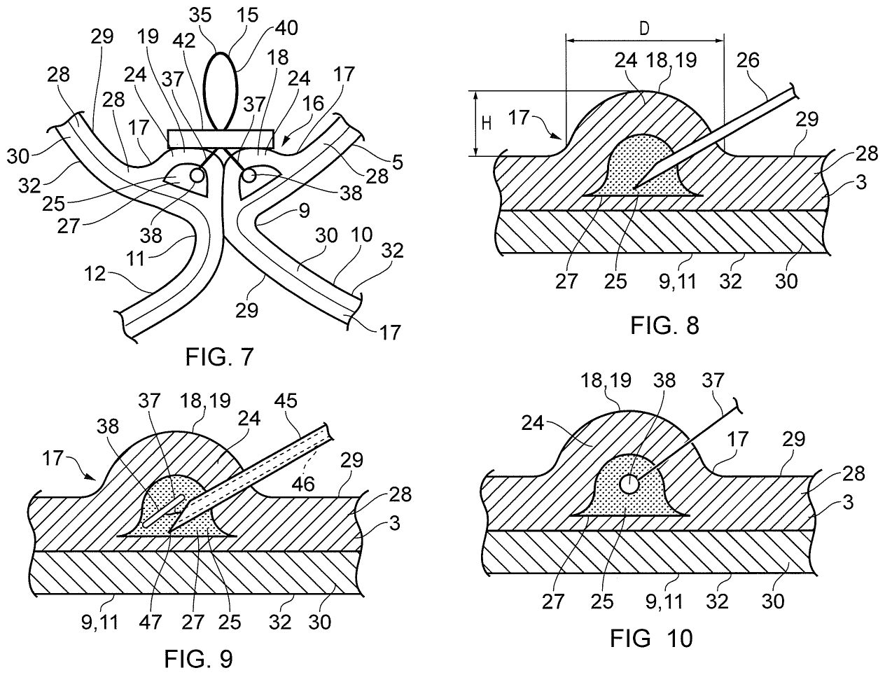

Referring to the drawings and initially to FIGS. 1 to 13 thereof, a method for suturing first and second portions of a wall of a vessel together in the body of a human or animal subject will now be described. In this embodiment of the invention the vessel comprises a stomach indicated generally by the reference numeral 1 of a human subject 2, in which a sleeve gastroplasty procedure is to be carried out by the method according to the invention whereby two portions of the wall 3 of the stomach 1 are sutured together, to form a gastric sleeve 5 extending through the stomach 1 and a lower redundant portion 7. The two portions of the stomach 1 which are sutured together are a first portion 9 of a front wall 10 of the stomach 1 and a second portion 11 of a rear wall 12 of the stomach 1. The first and second portions 9 and 11 of the stomach wall 3 are secured together by a plurality of sutures 15 which are inserted into respective pairs 16 of first mounds 18 and second mounds 19, formed at sites 17 on the respective first portion 9 and second portion 11 of the wall 3, as will be described in detail below, see FIGS. 1 to 3.

In this embodiment of the invention the sleeve gastroplasty procedure is carried out endoscopically internally in the stomach 1. An endoscope 21 is inserted into the subject 2 orally and through the oesophagus 23 into the stomach 1 of the subject 2, see FIG. 13. The stomach 1 is insufflated through an insufflating channel (not shown) of the endoscope 21, and instruments for carrying out the sleeve gastroplasty procedure are inserted into the stomach 1 through an instrument channel (also not shown) in the endoscope 21, as will be described below. Such endoscopes as the endoscope 21 will be well known to those skilled in the art, and further description of the endoscope should not be required.

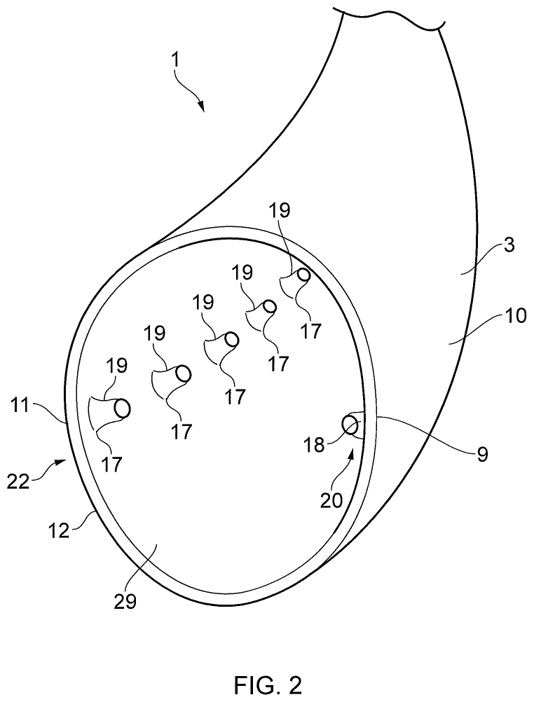

In carrying out the method according to the invention, the first and second mounds 18 and 19 are initially formed on the first and second portions 9 and 11 in the wall 3 of the stomach 1. The first mounds 18 are formed spaced apart along a first row 20 on the first portion 9 of the front wall 10 of the stomach 1, and the second mounds 19 are formed spaced apart in a second row 22 on the second portion 11 of the rear wall 12, see FIGS. 1 and 2. The first mounds 18 are substantially equi-spaced apart in the first row 20, and the second mounds 19 are substantially equi-spaced apart in the second row 22. The spacing between the first mounds 18 in the first row 20, and the spacing between the second mounds 19 in the second row 22 is substantially similar, so that each first mound 18 corresponds with one of the second mounds 19 to form a pair thereof for suturing together by a corresponding one of the sutures 15.

The first and second rows 20 and 22 of the first and second mounds 18 and 19 are sequentially formed, and the first mounds 18 in the first row 20 are sequentially formed, and the second mounds 19 in the second row 20 are also sequentially formed. Although, in some embodiments of the invention the first and second mounds of each one of the pairs 16 thereof may be sequentially formed before the next pair 16 of first and second mounds are formed, and as each one of the first and second mounds of each pair 16 thereof is formed, the suture may be inserted into the just formed mound before the next mound of the pair 16 thereof is formed.

Each one of the first and second mounds 18 and 19 is formed by injecting an injectable fluid into a submucosal layer 28 of the wall 3 of the stomach 1 through an interior surface 29 of the wall 3 of the stomach 1 adjacent the site 17, at which the corresponding one of the first or second mounds 18 or 19 is to be formed to form a pocket 27 therein, see FIG. 8. In this embodiment of the invention the injectable fluid comprises a viscous liquid, in this case a viscous bulking solution 25. The viscous bulking solution 25 is injected into the submucosal layer 28 by a needle 26 entered into the stomach 1 through the instrument channel of the endoscope 21. The viscous bulking solution 25 when injected into the submucosal layer 28 forms the pocket 27 beneath the site 17 at which the mound 18 or 19 is to be formed, see FIG. 8. The formation of the pocket 27 in the submucosal layer 28 deforms the submucosal layer 28, thereby resulting in a portion 24 of the submucosal layer 28 rising above the pocket 27 to in turn form the corresponding mound 18 or 19.

In embodiments of the invention where a viscous bulking solution is injected to form the pockets 27, any suitable viscous bulking solution may be used. However, it is desirable that the viscous bulking solution 25 be of viscosity such that the viscous bulking solution 25 remains as a homogenous mass in the pocket 27, with minimal dispersion from the pocket 27, for a predefined time period sufficient to enable insertion of the suture 15 into the corresponding first or second mound 18 or 19, and preferably, until all the sutures 15 have been inserted into the respective first and second mounds 18 and 19 of the corresponding pair thereof. In some embodiments of the invention it may be desirable that the viscous bulking solution 25 should remain in a homogenous mass in the pockets 27 until the sleeve gastroplasty procedure has been completed, which may be up to 30 minutes. Although, in general, it may be sufficient for the viscous bulking solution 25 to remain in the pocket 27 as a homogenous mass with minimal dispersion into the submucosal layer 28 for a predefined time period in the range of 5 minutes to 30 minutes. Suitable viscous bulking solutions are sometimes referred to as lifting agents. One such suitable lifting agent is a lifting agent sold under the trade mark ORISE gel by Boston Scientific, and another suitable lifting agent is that sold under the trade mark ELEVIEW by Aries Pharmaceutical.

A sufficient amount of the viscous bulking solution 25 is injected into the submucosal layer 28 at each site 17 such as to form the pocket 27 of an appropriate size in order to form the corresponding one of the first and second mounds 18 or 19 of the desired size. In this embodiment of the invention the quantity of the viscous bulking solution 25 injected into the submucosal layer 28 at each site 17 is approximately 1 ml to 2 ml, which it has been found is sufficient to produce the first and second mounds 18 and 19 to be of substantially circular dome shape of diameter D when viewed in plan, in the range of 5 mm to 10 mm, and of height H above the interior surface 29 of the wall 3 adjacent the corresponding site 17 in the range of 5 mm to 10 mm, see FIG. 8, although in some embodiments of the invention up to 5 ml or more of the viscous bulking solution may be required to form each mound 18 or 19.

In some embodiments of the invention, the viscous bulking solution 25 may be injected into the wall 3 of the stomach 1 adjacent a boundary between the submucosal layer 28 and an adjacent muscular layer 30 of the wall 3 in order to form the pockets 27 at the boundary between the submucosal layer 28 and the muscular layer 30.

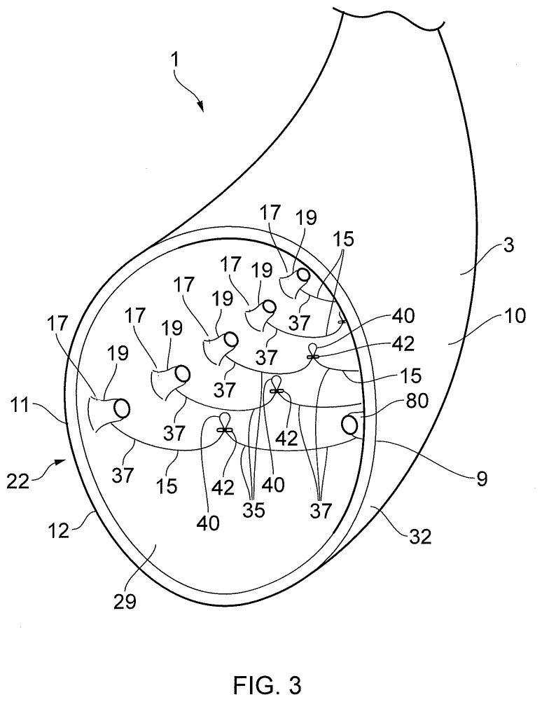

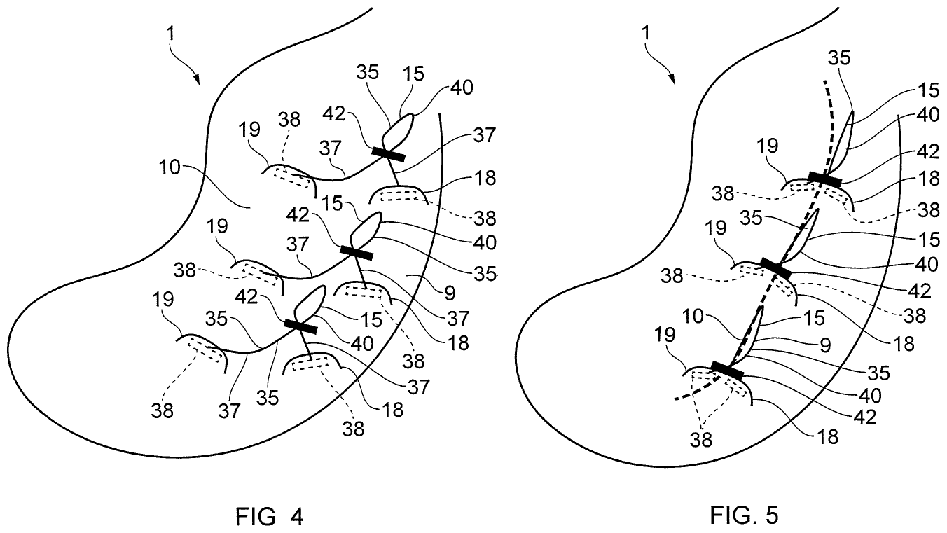

On the completion of the formation of the first and second mounds 18 and 19 in the first and second rows 20 and 22, respectively, on the first and second portions 9 and 11 of the wall 3 of the stomach 1, the needle 26 is withdrawn from the stomach 1 through the instrument channel of the endoscope 21, and the sutures 15 are then sequentially inserted into the respective pairs 16 of the first and second mounds 18 and 19, see FIGS. 3 and 4. The sutures 15 are then tightened for drawing the first and second mounds 18 and 19 of the respective pairs 16 tightly together, for in turn drawing the first and second portions 9 and 11 of the front and rear walls 10 and 12 together to promote knitting of the first and second portions 9 and 11 together adjacent the first and second rows 20 and 22 of the first and second mounds 18 and 19, see FIGS. 1, 5 and 7.

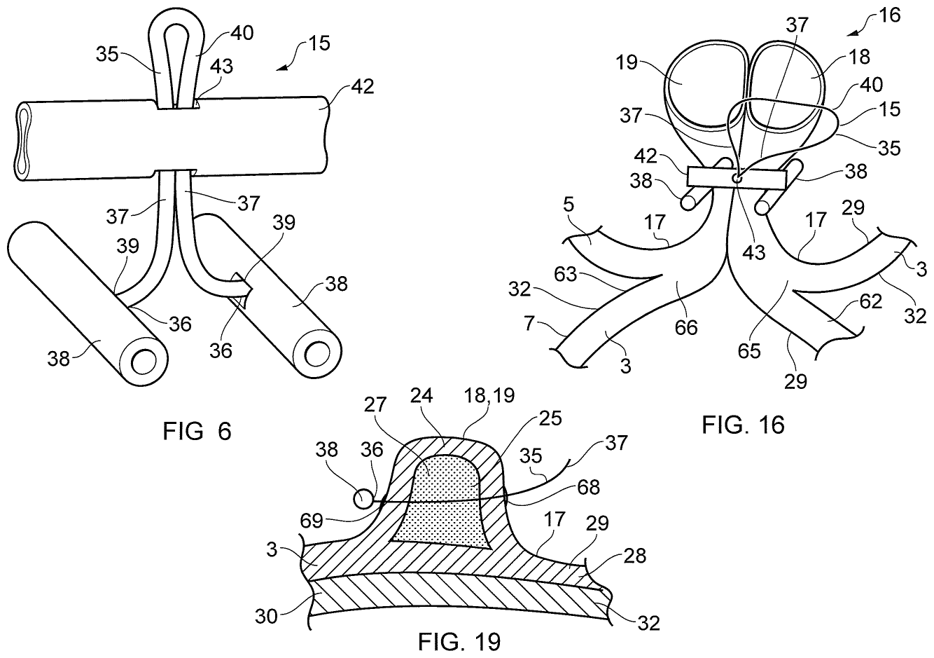

Before describing the insertion of the sutures 15 into the first and second mounds 18 and 19 of the respective pairs 16 thereof, one of the sutures 15 will first be described with reference to FIG. 6. Each suture 15 comprises an elongated suture thread 35 terminating in respective opposite ends 36 thereof in anchor elements, in this embodiment of the invention anchor bars 38. The suture thread 35 is secured to each anchor bar 38 adjacent a mid-point 39 of the anchor bar 38. Legs 37 of the suture thread 35 extend from each anchor bar 38 to form a loop 40. A cinch clip 42 having a loop accommodating bore 43 extending therethrough is located on the loop 40 with the loop 40 extending through the loop accommodating bore 43. The cinch clip 42 engages the loop 40 with a tight sliding fit, and is urgeable along the loop 40 towards the anchor bars 38 to reduce the effective length of the suture thread 35 between the anchor bars 38 for tightening the suture 15, to in turn draw the corresponding pair 16 of the first and second mounds 18 and 19 together. The tightness of the sliding fit of the cinch clip 42 on the legs 37 of the suture thread 35 forming the loop 40 is such as to permit sliding of the cinch clip 42 along the loop 40 towards the anchor bars 38, but is tight enough to resist sliding of the cinch clip 42 in the reverse direction from the anchor bars 38 against any force induced in the suture thread 35 in the legs 37 thereof by the front and rear walls 10 and 12 of the stomach 1 tending to return to their original state.

Each suture 15 is inserted into the corresponding pair 16 of the first and second mounds 18 and 19 by a suturing instrument, for example, a suturing instrument of the type disclosed in PCT Published Specification No. WO 2021/165945, the disclosure of which is incorporated herein by reference. The suturing instrument is entered into the stomach 1 through the instrument channel of the endoscope 21. Only a cannula 45 of the suturing instrument thereof is illustrated in FIG. 9. The cannula 45 terminates in a pointed piercing tip 47, and a bore 46 extends through the cannula 45. The suture 15 is located in the bore 46 adjacent the pointed tip 47, with the anchor bars 38 of the suture 15 sequentially located in the bore 46, as described in PCT Published Specification No. WO 2021/165945, so that the anchor bars 38 of the suture 15 may be sequentially discharged from the cannula, and in turn sequentially inserted into the respective first and second mounds 18 and 19 of the corresponding pair 16 thereof.

Initially, the first mound 18 of the first pair 16 of the first and second mounds 18 and 19 is pierced by the pointed tip 47 of the cannula 45 of the suturing instrument, and the pointed tip 47 of the cannula 45 is urged into the pocket 27 of the first mound 18. With the pointed tip 47 located in the pocket 27, the suturing instrument is operated to discharge the first one of the two anchor bars 38 from the bore 46 of the cannula 45 through the pointed tip 47 thereof, thereby depositing the first one of the anchor bars 38 in the pocket 27 of the first mound 18, see FIG. 9. The cannula 45 is withdrawn from the first mound 18, with the leg 37 of the suture thread 35, which is extending from the anchor bar 38 in the pocket 27, through the portion 24 of the submucosal layer 28 forming the first mound 18.

The second mound 19 of the pair 16 of the first and second mounds 18 and 19 is then pierced by the pointed tip 47 of the cannula 45, and the pointed tip 47 thereof is urged into the pocket 27 of the second mound 19. The second one of the two anchor bars 38 of the suture 15 is then discharged from the cannula 45 into the pocket 27 of the second mound 19 in a similar manner as the first anchor bar 38 is discharged into the pocket 27 of the first mound 18. The cannula 45 is then withdrawn from the second mound 19 with the leg 37 of the suture thread 35 extending from the second one of the anchor bars 38 in the pocket 27 through the portion 24 of the second mound 19.

As will be understood by those skilled in the art from the description of the suturing instrument in PCT Published Specification No. WO 2021/165945, the cinch clip 42 on the loop 40 of the suture thread 35 of the suture 15 will have been discharged into the stomach from the bore 46 of the cannula 45, or from a distal end of a bore of a sleeve in which the cannula 45 is slideable, prior to the piercing of the first mound 18 of the pair 16 thereof. Once the cannula 45 has been withdrawn from the second mound 19 of the pair thereof, the cannula is withdrawn from the stomach through the instrument channel of the endoscope 21 to be recharged with the next suture. On being recharged with the next suture 15, the cannula 45 of the suturing instrument is returned to the stomach 1 through the instrument channel of the endoscope 21 for inserting the next one of the sutures 15 into the next pair 16 of the first and second mounds 18 and 19, and so on until the sutures 15 have been inserted into the first and second mounds 18 and 19 of all of the pairs 16 thereof.

On completion of insertion of the sutures 15 into the respective pairs 16 of the first and second mounds 18 and 19 thereof, the sutures 15 are sequentially tightened to draw the first and second mounds 18 and 19 of the respective pairs 16 thereof tightly together, for in turn drawing the first and second portions 9 and 11 of the front and rear walls 10 and 12, respectively, of the stomach 1 tightly together along the first and second rows 20 and 22 of the first and second mounds 18 and 19 to promote knitting thereof. Each suture 15 is tightened by urging the cinch clip 42 along the loop 40 of the suture thread 35 of the suture 15 for drawing the anchor bars 38 of the suture 15 together, for in turn drawing the first and second mounds 18 and 19 of the corresponding pair 16 thereof tightly together.

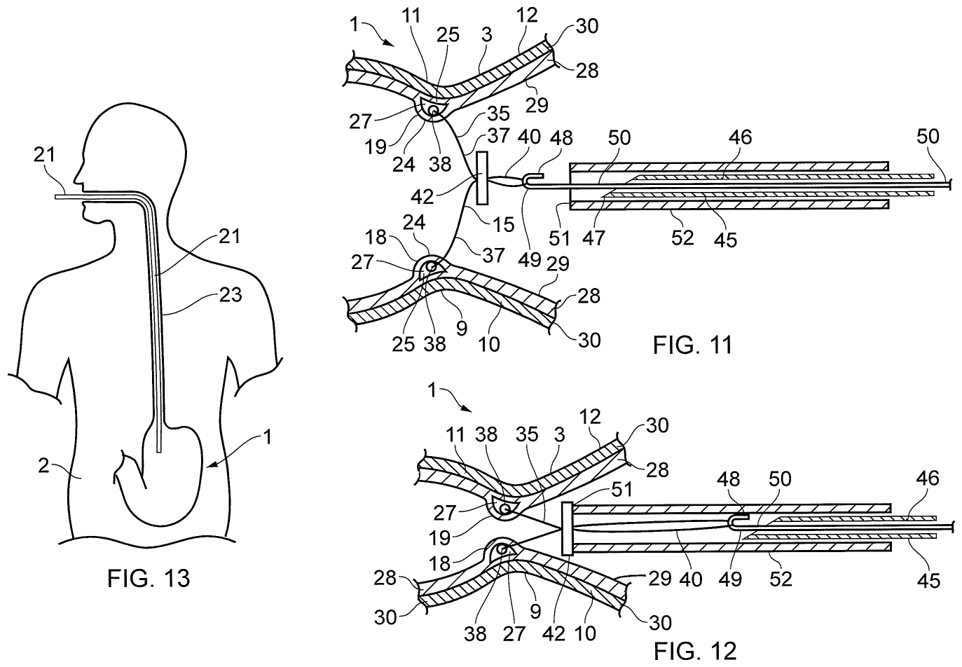

Any suitable means for urging the cinch clips 42 along the loops 40 of the suture threads 35 of the respective sutures 15 may be used. In this embodiment of the invention the sutures 15 are tightened by the suturing instrument disclosed in PCT Specification No. WO 2021/165945. Only a distal part of the suturing instrument for tightening the sutures 15 is illustrated in FIGS. 11 and 12. A hook 48 adjacent the distal end 49 of a pusher element 50 of the suturing instrument, which extends through the cannula bore 46 of the cannula 45 is urged outwardly of the cannula 45 and through a distal end 51 of a sleeve 52 within which the cannula 45 is slideable. The hook 48 is engaged with the loop 40 of the suture 15 which is to be tightened, see FIG. 11. With the hook 48 engaging the loop 40 of the corresponding suture 15, the pusher element 50 along with the hook 48 is withdrawn with the cannula 45 into the outer sleeve 52 until the cinch clip 42 abuts the distal end 51 of the outer sleeve 52, see FIG. 12. Further urging of the loop 40 into the outer sleeve 52 by the hook 48, with the cinch clip 42 abutting the distal end 51 of the outer sleeve 52 results in the cinch clip 42 being urged along the loop 40 of the suture 15 towards the anchor bars 38 for tightening the suture 15, see again FIG. 12. The tightening of the suture 15 by urging the cinch clip 42 along the loop 40 of the suture thread 35 towards the anchor bars 38 for tightening a suture, similar to the suture 15, is described in PCT Specification No. WO 2021/165945.

Alternatively, the sutures 15 may be tightened by any suitable suture securing instrument of the type also disclosed in PCT Specification No. WO 2021/165945. Thereafter, the suturing instrument or the suture securing instrument, as the case may be, is withdrawn from the stomach 1 through the instrument channel of the endoscope 21, and the insufflating gas is withdrawn from the stomach 1.

In carrying out the endoscopic sleeve gastroplasty procedure the endoscope 21 is inserted orally through the oesophagus 23 into the stomach 1. The stomach 1 is insufflated by a suitable insufflating gas delivered into the stomach 1 through an insufflating channel (not shown) of the endoscope 21. With the stomach 1 insufflated, the injecting needle 26 connected to a source (not shown) of the viscous bulking solution 25 is inserted through the instrument channel (not shown) of the endoscope 21 into the stomach 1. The injecting needle 26 is manoeuvred within the stomach 1 and moved sequentially from one site 17 to the next site 17. At each site 17, the appropriated amount of the viscous bulking solution 25 is injected through the interior surface 29 of the wall 3 of the stomach 1 into the submucosal layer 28 to form the corresponding pocket 27, and in turn the corresponding one of the first and second mounds 18 and 19 in the corresponding one of the first and second portions 9 and 11, respectively, of the wall 3. On completion of the formation of the first and second mounds 18 and 19, the injecting needle 26 is withdrawn through the instrument channel of the endoscope 21.

The suturing instrument charged with one of the sutures 15, is urged into the stomach 1 through the instrument channel (not shown) of the endoscope 21. The suture 15 is then inserted sequentially into the first and second mounds 18 and 19 of the first pair 16 thereof by inserting the anchor bars 38 of the suture 15 into the pockets 27 of the respective first and second mounds 18 and 19 of the pair 16 thereof, as already described. The suturing instrument is then withdrawn through the instrument channel of the endoscope 21 and recharged with the next suture 15. Thereafter, sutures 15 are inserted into the first and second mounds 18 and 19 of the respective pairs 16 thereof sequentially in a similar manner until sutures 15 have been inserted into all the pairs 16 of the first and second mounds 18 and 19.

Once the sutures 15 have been inserted into the respective remaining pairs 16 of the first and second mounds 18 and 19 with the anchor bars 38 of the sutures 15 located within the pockets 27 of the first and second mounds 18 and 19 of the pairs 16 thereof, the suturing instrument is then used to tighten the sutures 15 of the respective pairs 16 of the first and second mounds 18 and 19, in order to draw the first and second mounds 18 and 19 of the pairs 16 thereof together, and in turn the first and second portions 9 and 11 of the front and rear walls 10 and 12, respectively, together, to promote knitting thereof, as already described.

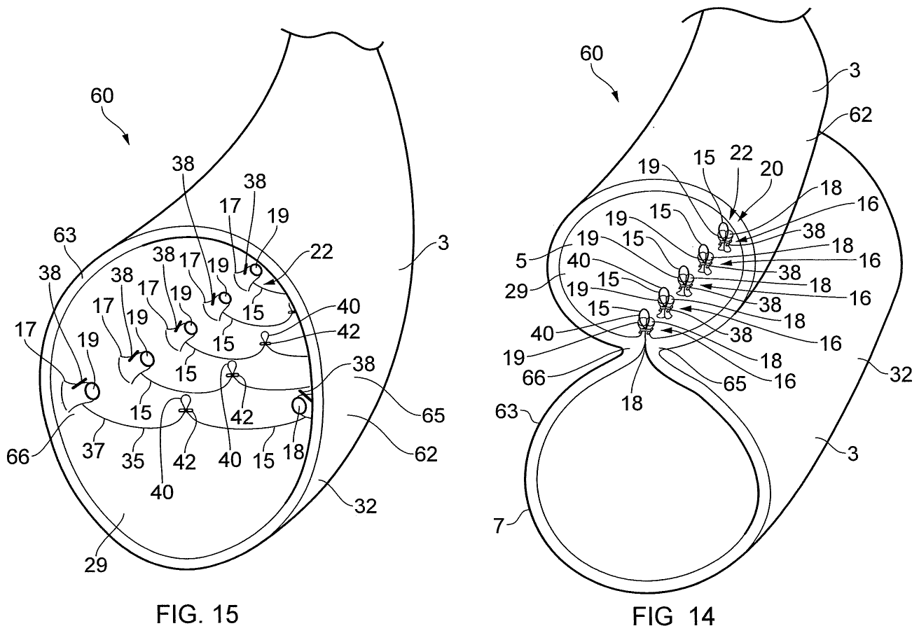

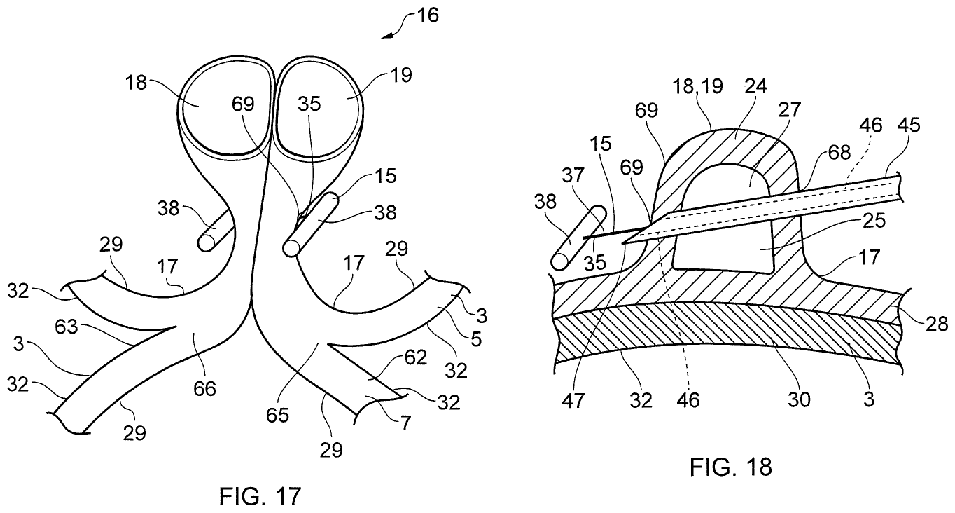

Referring now to FIGS. 14 to 19, a method for suturing first and second portions of a wall of a vessel together in the body of a human or animal subject according to another embodiment of the invention will now be described. In this embodiment of the invention, like the embodiment of the invention described with reference to FIGS. 1 to 13, the vessel comprises a stomach indicated generally by the reference numeral 60 of a human subject, in which an endoscopic sleeve gastroplasty procedure is also to be carried out by the method according to this embodiment of the invention to form a gastric sleeve 61 extending through the stomach 60. The method according to this embodiment of the invention is substantially similar to the method described with reference to FIGS. 1 to 13 in which a sleeve gastroplasty procedure has been described, and similar components are identified by the same reference numerals. The only difference between the method of this embodiment of the invention and that described with reference to FIGS. 1 to 13 is in the actual suturing of the front and rear walls 62 and 63 of the stomach 60 together.

In the method according to this embodiment of the invention first and second mounds 18 and 19, which are similar to the first and second mounds 18 and 19 formed on the interior surface 29 of the front and rear walls 10 and 12 of the stomach 1, are formed on first and second portions 65 and 66, respectively, of the front and rear walls 62 and 63, respectively, of the stomach 60. The first and second mounds 18 and 19 formed on the first and second portions 65 and 66 of the front and rear walls 62 and 63 are formed in an identical manner as the first and second mounds 18 and 19 are formed on the interior surface 29 of the front and rear walls 10 and 12 of the stomach 1. The first and second mounds 18 and 19 are formed by injecting the viscous bulking solution 25 into the submucosal layer 28 at sites 17 of the first and second portions 65 and 66 of the front and rear walls 62 and 63 of the stomach 60 in order to form respective pockets 27 adjacent the sites 17 at which the first and second mounds 18 and 19 are to be formed.

Once the first and second mounds 18 and 19 have been formed, the sutures 15, described with reference to FIG. 6 are then inserted sequentially into the first and second mounds 18 and 19 of the respective pairs 16 thereof. However, in this embodiment of the invention instead of the anchor bars 38 of the respective sutures 15 being inserted into the pockets 27 of the corresponding ones of the first and second mounds 18 and 19, the cannula 45 of the suturing instrument disclosed in PCT Specification No. WO 2021/165945 is urged to pierce transversely through the respective first and second mounds 18 and 19 of each pair 16 thereof from an entry side 68 thereof to an exit side 69 thereof as illustrated in FIG. 18.

On the cannula 45 having completely pierced through the first one of the first and second mounds 18 and 19 of the corresponding pair 16 thereof, the first one of the anchor bars 38 of the suture 15 is discharged from the bore 46 of the cannula 45 on the exit side 69 of the first mound 18, as illustrated in FIG. 18. The cannula 45 is withdrawn from the first mound 18 with the suture thread 35 extending from the anchor bar 38 through the first mound 18 from the exit side 69 to the entry side 68 thereof, see FIG. 19. The suture 15 is then inserted in the second mound 19 of the pair 16 of the first and second mounds 18 and 19 by the cannula 45, in a similar manner as described with reference to the first mound 18, with the anchor bar 38 located on the exit side 69 of the second mound 19, and the cannula 45 is withdrawn with the suture thread 35 extending from the anchor bar 38 through the second mound 19 from the exit side 69 to the entry side 68 thereof, see FIG. 15. With the suture 15 inserted in the first and second mounds 18 and 19 of the corresponding pair 16 thereof, the cannula 45 is withdrawn through the instrument channel of the endoscope 21 and recharged with the next suture 15. The sutures 15 are sequentially inserted in the pairs 16 of the first and second mounds 18 and 19 until the sutures 15 have been inserted into the remaining pairs 16 of the first and second mounds 18 and 19 with the anchor bars 38 of the suture 15 located on the exit side 69 of the respective first and second mounds 18 and 19 of the pair 16 thereof, and with the legs 37 of the suture thread 35 extending from the respective anchor bars 38 through the corresponding ones of the first and second mounds 18 and 19, see FIG. 15.

Once the sutures 15 have been inserted into the respective pairs 16 of the first and second mounds 18 and 19, the sutures are tightened by urging the cinch clips 42 along the loops 40 of the suture thread 35 towards the anchor bars 38 in a similar manner as described with reference to the tightening of the sutures 15 with respect to the method described with reference to FIGS. 1 to 13. As the sutures 15 of the respective pairs 16 of the first and second mounds 18 and 19 are tightened, the first and second mounds 18 and 19 are drawn together as illustrated in FIGS. 14, 16 and 17. By drawing the first and second mounds 18 and 19 of the pairs 16 thereof together, the first and second portions 65 and 66 of the front and rear walls 62 and 63, respectively, are drawn together along the first and second rows 20 and 22 of the first and second mounds 18 and 19, as illustrated in FIGS. 14, 16 and 17.

Otherwise, the suturing of the front and rear walls 62 and 63 of the stomach 60 together to form the gastric sleeve 61 in the endoscopic sleeve gastroplasty procedure is similar to the method for carrying out the endoscopic sleeve gastroplasty procedure described with reference to FIGS. 1 to 13 to form the gastric sleeve 5.

While the method has been described for use in carrying out a sleeve gastroplasty procedure, it will be readily apparent to those skilled in the art that the method according to the invention may be used for suturing any two portions of a wall of any vessel, lumen or organ together, and furthermore, it will be readily apparent to those skilled in the art that the method according to the invention may be used for suturing two parts of a vessel, lumen or organ whereby the method is carried out within the vessel, lumen or organ, or is carried out externally of the vessel, lumen or organ. For example, the method may be used for suturing two portions of a wall of a vessel, lumen or organ together externally of the vessel, lumen or organ in, for example, a cavity in which the vessel, lumen or organ is located. For example, in the case of a vessel, lumen or organ which is located in the peritoneal cavity, it is envisaged that the method could be carried out laparoscopically in the peritoneal cavity in which the vessel, lumen or organ, the two portions of which are to be sutured together, is located.

It will be appreciated that while a specific suturing instrument has been described for suturing the two parts of the vessel wall together, any other suitable suturing instrument may be used. It will also be appreciated that while sutures of a specific type have been described for use in the method, any other suitable type of suture may be provided for inserting into the first and second mounds of each pair thereof, and for drawing the first and second mounds of the respective pairs together.

It will also be appreciated that while the method has been described whereby the first mounds of the first row have been formed sequentially, and the second mounds of the second row have been described as being formed sequentially before insertion of the sutures, it is envisaged that in some embodiments of the invention each time either a pair or one of a pair of the first and second mounds have been formed, the suture may be inserted into each one of the first and second mounds of the pair thereof as each mound has been formed, or the suture may be inserted into the first and second mounds of the corresponding pair thereof sequentially after the pair of the mounds have been formed before the next pair of first and second mounds are formed.

While in the embodiments of the invention described, the pockets have been described as being formed in the submucosal layer, in some embodiments of the invention it is envisaged that the pockets may be formed between the submucosal layer and a muscular layer of the wall of the vessel.

While the injectable fluid has been described as comprising a viscous bulking solution, while this is desirable, it is not essential. In some embodiments of the invention a less viscous fluid may be suitable. Indeed, in some embodiments of the invention the injectable fluid may be water or a saline solution. The choice of the injectable fluid will to some extent be dependent on the site at which the pocket is to be formed, the nature of the vessel wall, and the length of time between the formation of the mound, and the insertion of the suture into or through the mound. Since in general, it is believed that a less viscous injectable fluid will disperse into the submucosal layer or between the submucosal layer and the muscular layer of the vessel wall quicker than a more viscous injectable fluid would disperse, the viscosity of the injectable fluid, will most likely be dependent on the time between the formation of the mound and the insertion of the suture into or through the mound. It is believed that if the suture is to be inserted into or through a pair of the first and second mounds immediately after the formation of the mounds, a saline solution would be suitable for forming the pockets in the submucosal layer.

Additionally, while specific injectable viscous bulking solutions have been described, any other suitable viscous bulking solutions may be used.

While a specific type of suture has been described, it will be readily apparent to those skilled in the art that any other suitable suture may be used.

1. A method for suturing a first portion of a wall of a vessel in a cavity in the body of a human or animal subject to a second portion of the wall of the vessel either interiorly in the interior of the vessel, or exteriorly of the vessel in the cavity, the method comprising:
forming at least one first mound on a site of the first portion of the vessel wall,
forming at least one second mound on a site of the second portion of the vessel wall,
each one of the first and second mounds being formed by forming a pocket in a submucosal layer of the vessel wall or between the submucosal layer and a muscular layer of the vessel wall adjacent the site at which the corresponding one of the first and second mounds is to be formed,
inserting at least one suture into or through a corresponding pair of the at least one first mound, and the at least one second mound, and
tightening the suture to draw the corresponding pair of the first and second mounds together.

2. A method as claimed in claim 1 in which each pocket is formed by injecting an injectable fluid into the submucosal layer of the vessel wall or between the submucosal layer and the muscular layer of the vessel wall where the pocket is to be formed.

3. A method as claimed in claim 2 in which the injectable fluid comprises an injectable liquid.

4. A method as claimed in claim 2 in which the injectable fluid comprises a viscous solution.

5. A method as claimed in claim 2 in which the viscosity of the injectable liquid is sufficient to minimise dispersal of the injectable fluid from the corresponding pocket for a predefined time period at least sufficient to allow the suture to be inserted into the mound.

6. A method as claimed in claim 2 in which the quantity of the injectable fluid injected into the submucosal layer or between the submucosal layer and the muscular layer of the corresponding one of the first and second portions of the vessel wall to form the pocket of the corresponding one of the first or second mounds lies in the range of 1 ml to 10 ml.

7. A method as claimed in claim 1 in which a plurality of the first mounds are formed spaced apart on the respective sites on the first portion of the vessel wall, and a plurality of the second mounds are formed spaced apart on the respective sites on the second portion of the vessel wall.

8. A method as claimed in claim 1 in which each one of the first and second mounds comprises a substantially circular mound when viewed in plan.

9. A method as claimed in claim 1 in which each one of the first and second mounds comprises an elongated mound extending along the corresponding one of the first and second portions of the vessel wall.

10. A method as claimed in claim 9 in which a plurality of sutures are inserted into or through each one of the first and second mounds when each one of the first or second mounds comprises an elongated mound.

11. A method as claimed in claim 1 in which the height of each one of the first and second mounds from a surface of the vessel wall adjacent the site at which the mound is formed lies in the range of 5 mm to 10 mm.

12. A method as claimed in claim 1 in which each suture comprises a suture thread terminating at respective opposite ends thereof in respective anchor elements, the suture thread defining a loop formed by two legs of the suture thread extending from the respective anchor elements, the loop extending through a cinch clip, the cinch clip being urgeable along the loop towards the anchor elements for reducing the effective length of the suture thread extending between the anchor elements to tighten the suture.

13. A method as claimed in claim 12 in which the cinch clip of each structure is configured to be a tight sliding fit on the loop sufficient to resist movement of the cinch clip along the loop in a direction away from the anchor elements.

14. A method as claimed in claim 12 in which each suture is inserted into the first and second mounds of the corresponding pair thereof by inserting the anchor elements into the pockets of the respective first and second mounds of the pair thereof with the legs of the suture thread extending from the anchor elements through the corresponding mounds.

15. A method as claimed in claim 12 in which each suture is inserted into the first and second mounds of the corresponding pair thereof by urging the anchor elements of the suture through the respective ones of the first and second mounds from an entry side of the corresponding ones of the first and second mounds to an exit side thereof with the anchor elements located on the exit side of the corresponding ones of the first and second mounds and the legs of the suture thread extending from the anchor elements through the corresponding ones of the first and second mounds from the exit side thereof to and through the entry side thereof.

16. A method as claimed in claim 12 in which each suture is tightened by urging the cinch clip thereof along the loop formed by the suture thread thereof towards the anchor elements for drawing the first and second mounds of the corresponding pair thereof together.

17. A method as claimed in claim 1 in which the suturing of the first and second portions of the vessel is carried out endoscopically internally within the vessel.

18. A method as claimed in claim 1 in which the method is carried out endoscopically by inserting an endoscope into the vessel orally, rectally or vaginally.

19. A method as claimed in claim 1 in which the suturing of the first and second portions of the vessel together is carried out in the cavity externally of the vessel.

20. A method for carrying out a sleeve gastroplasty procedure in the stomach of a human or animal subject in which first and second portions of the wall of the stomach are sutured together to form a gastric sleeve by the method as claimed in claim 1.
